(19) United States
(12) Patent Application Publication
(10) Pub. No.: US 2007/0005085 A1
(43) Pub. Date: Jan. 4, 2007

(54) Subcutaneous tunneller

(75) Inventor: Anthony Lambert, Plymouth (GB)

Correspondence Address: BROOKS KUSHMAN P.C., 1000 TOWN CENTER, TWENTY-SECOND FLOOR, SOUTHFIELD, MI, 48075, US

(73) Assignee: LAMBERT SURGICAL DEVELOPMENTS LTD, Coventry (GB)

(21) Appl. No.: 10/595,027

(22) PCT Filed: Jun. 23, 2004
(86) PCT No.: PCT/GB04/02688

§ 371 Date: Sep. 1, 2006

(30) Foreign Application Priority Data
Jun. 24, 2003 (GB) 03146891

(51) Int. Cl. A61B 17/32

(52) U.S. Cl. 606/167

(57) ABSTRACT

The invention provides an orchidopexy tunneller (10) for us in orchidopexy surgery. The tunneller comprises an arcuate elongate body (12) having a tunneling head (14), and attachment means (16) at the opposite end thereof for attachment with a testicle to be repositioned in the human body by the tunneller during orchidopexy surgery. The attachment means is preferably capable of being secured to the testis by means of a surgical stitch, and may comprise an aperture formed in the end of the body 12 for holding the stitch. The invention also provides a unidirectional orchidopexy surgical procedure wherein an incision (30) is made in the abdominal wall of a patient in the region of the undescended testis so that the testis can be located externally of the patient's body. The tunneller head is inserted in the incision (30) then moved to a position within the scrotum (38) to form a subcutaneous tunnel and scrotal pouch (dartos pouch) (40). A second incision (42) is formed in the scrotum in the region of the tunneller head so that the tunneller may be pulled through the patient's body with the undescended testis (34) attached to the other end of the tunneller by means of a surgical stitch. The surgical procedure is completed by releasing the testis from the end of the tunneller and anchoring it to the scrotum prior to closing the incisions previously formed.

This invention relates to subcutaneous surgical tunnellers and in particular concerns a tunneller suitable for use in orchidopexy surgery to correct undescended testis in the human male body.

Cryptorchism (undescended testis) is a congenital condition where one or more testis fail to descend from the abdominal cavity into the scrotal sac. If left untreated this condition can result in an increased risk of testicular cancer. The condition is corrected using orchidopexy surgery first to dissect and mobilise the undescended testis with respect to the abdominal wall and then to grasp the testis and apply caudal traction to aid in dissection and move the testis into the scrotum so that it may be anchored in a subcutaneous scrotal pouch (dartos pouch). This procedure can involve a minimum of invasive surgery.

There is a requirement to simplify current surgical procedures so that trauma of the surrounding tissue is minimised and also to provide a quicker and more reliable procedure using the minimum of invasive instruments.

According to an aspect of the invention there is provided a one-piece orchidopexy tunneller comprising an elongate body having a tunnelling head at one head thereof and a testicle attachment means at the opposite end thereof for attachment to a testicle to be repositioned in the human body by means of a surgical stitch during orchidopexy surgery.

The orchidopexy tunneller of the present invention readily enables orchidopexy surgery to be undertaken by creating an incision in the abdominal wall in the region of the undescended testis, inserting the tunneller in the incision so formed and feeding the tunneller subcutaneously to the scrotum so that the tunneller head creates a dartos pouch for later anchoring the undescended testis within the scrotum. A further incision can then be made in the scrotum so that the tunneller head can be fed through the scrotum and out of the patient's body until the other end of the tunneller is positioned in the region of the first incision where the undescended testis is attached to that end of the tunneller so that further movement of the tunneller through the inguinal canal and scrotum applies caudal traction so that the testis follows the end of the tunneller into the scrotum where the testis can be detached from the end of the tunneller and anchored in the scrotum with the procedure being completed by closing the abdominal and scrotum incisions. This is a particularly effective surgical procedure since the tunneller is moved in a single direction only entering the first incision in the abdominal wall and exiting through the incision in the scrotum with the tunnel so formed allowing the testis to be repositioned. The tunneller of the present invention is particularly suitable for this surgical procedure since the tunnelling head is integrally formed at the end of the elongate body of the tunneller so that the tunnel can be formed by an appropriately sized tunneller head selected from a group of tunnellers having different size heads corresponding to different size testis thereby to correctly size the tunnel with respect to the testis to be moved therethrough.

Preferably the tunneller is capable of being secured to the testicle to be moved by means of a surgical stitch. It is sufficient to anchor the testis to the end of the tunneller opposite the tunneller head and this may conveniently be implemented during the surgical procedure by means of a stitch which engages a suitable feature on the end of the tunneller. The attachment means may comprise an aperture provided in the end of the body of the tunneller opposite the tunneller head. The aperture conveniently enables the stitch to be anchored to the end of the tunneller in a reliable manner.

Preferably the elongate body is arcuate so that the tunneller may be readily manipulated by the surgeon during the initial procedure of inserting the tunneller and forming the subcutaneous tunnel prior to attachment of the testis to the attachment means at the end of the body.

The invention also comprehends a uni-directional orchidopexy tunneller. In this respect it is to be understood that the term “uni-directional” refers to the one direction of movement of the tunneller through the patient's body. The invention also comprehends a kit of tunnellers having different size tunneller heads.

According to another aspect of the invention, there is provided a method of orchidopexy surgery on the male human or animal body, the said method comprising the steps of:

i) creating an incision in the abdominal wall of the body being treated in the region of the undescended testis;

ii) inserting a subcutaneous tunneller in the said incision and feeding the tunneller subcutaneously to the scrotum of the said body;

iii) forming a pouch in the scrotum to accommodate the said testis;

iv) creating an incision in the scrotum in the region of the said pouch;

v) attaching the said testis to the tunneller at a position remote from the head of the tunneller;

vi) feeding the tunneller and the said attached testis through the subcutaneous tunnel previously formed until the said testis is positioned in the region of the said pouch;

vii) detaching the said testis from the tunneller;

viii) closing the said incisions with the testis positioned in the said pouch.

The above aspects of the invention preferably provide a unidirectional orchidopexy surgical procedure.

An embodiment of the present invention will now be more particularly described, by way of example, with reference to the accompanying drawings, in which:

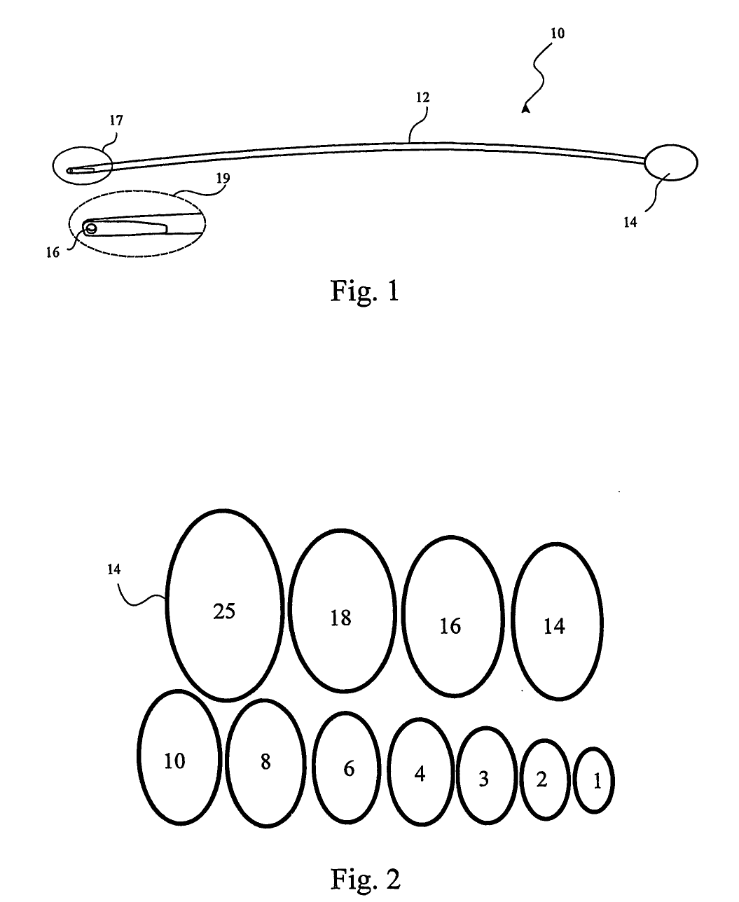

FIG. 1 is a schematic view of an orchidopexy surgical tunneller according to one arrangement of the present invention;

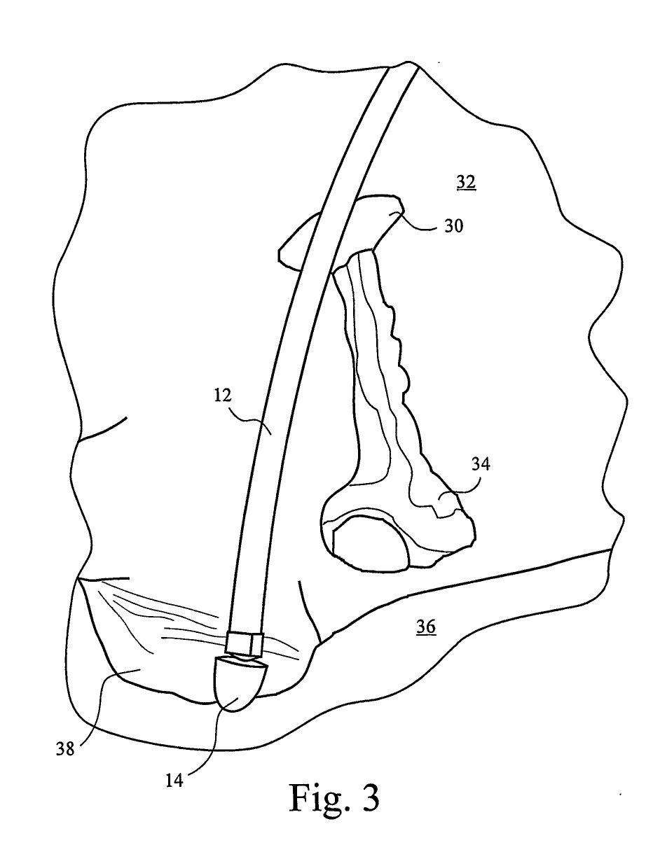

FIG. 2 is a schematic representation of the different size tunneller heads corresponding to different size testis;

FIGS. 3-7 show various stages of an orchidopexy surgical procedure using a surgical tunneller instrument according to an arrangement of the present invention.

Referring to FIG. 1 an orchidopexy tunneller 10 for use in orchidopexy surgery comprises elongate body 12 in the form of an arcuate rod. A tunneller head 14 is provided at one end of the elongate body 12 and a testis attachment means in the form of an aperture 16 is provided at the opposite end thereof. The tunneller head, elongate body and aperture 16 are integrally formed as a one-piece surgical instrument, in other embodiments the tunneller head may be detachably marked to the body 12 so that a set of different size heads may be selectively attached to the body 12. The invention contemplates a set of tunnellers having different size heads so that a tunneller may be selected from a set of different size tunnellers each having a head 14 corresponding to a different size of testis (see FIG. 2) so that an appropriate size tunneller may be used for the particular size of testis to be repositioned. In the illustration of FIG. 1 the end of the tunneller containing the aperture and encircled at 1 7 is shown in greater (enlarged) detail at 19.

The aperture 16 readily enables the testis to be moved by the tunneller to be attached to the end of the tunneller by means of a surgical stitch looped through the aperture 16. As can be seen in the drawing the aperture is formed in a tapered portion of the elongate body provided at the end of the body opposite the head 14.

The overall length dimension of the tunneller(s) of the illustrated embodiment present invention is approximately 300 mm. The tunneller(s) are preferable of a material suitable for surgical instruments, for example titanium or titanium alloy.

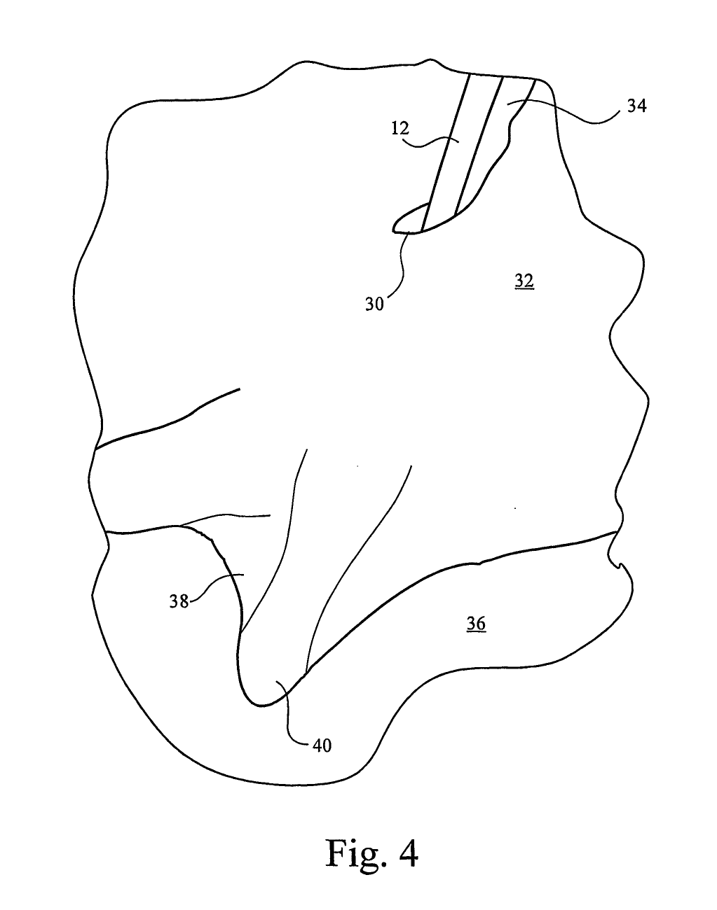

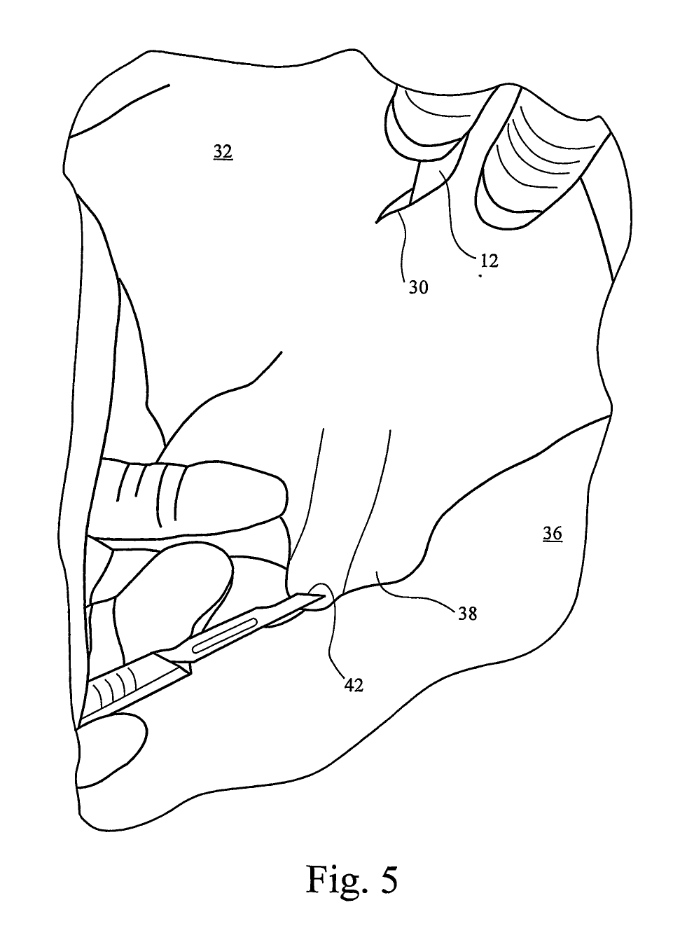

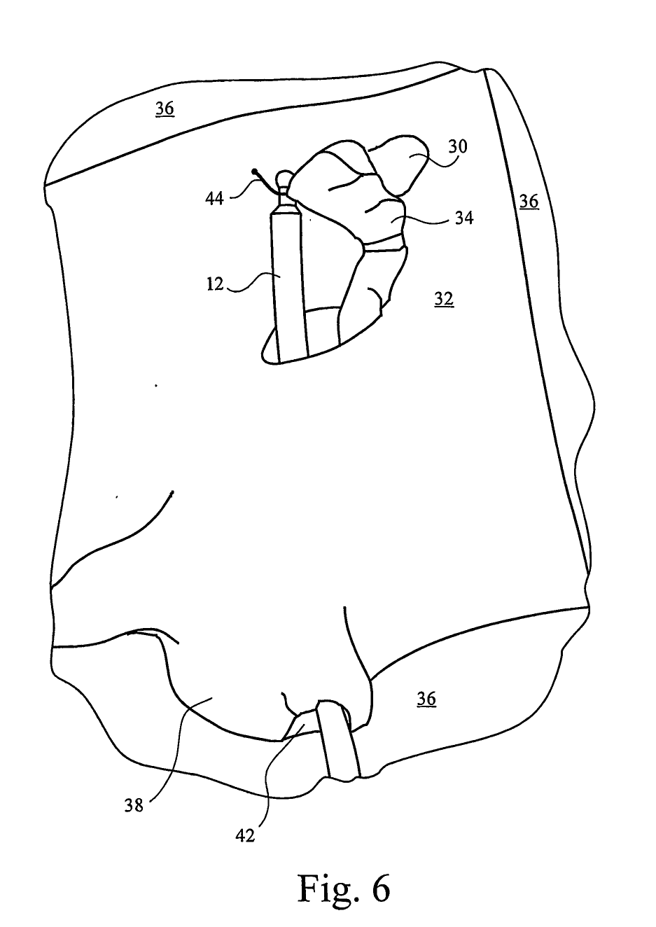

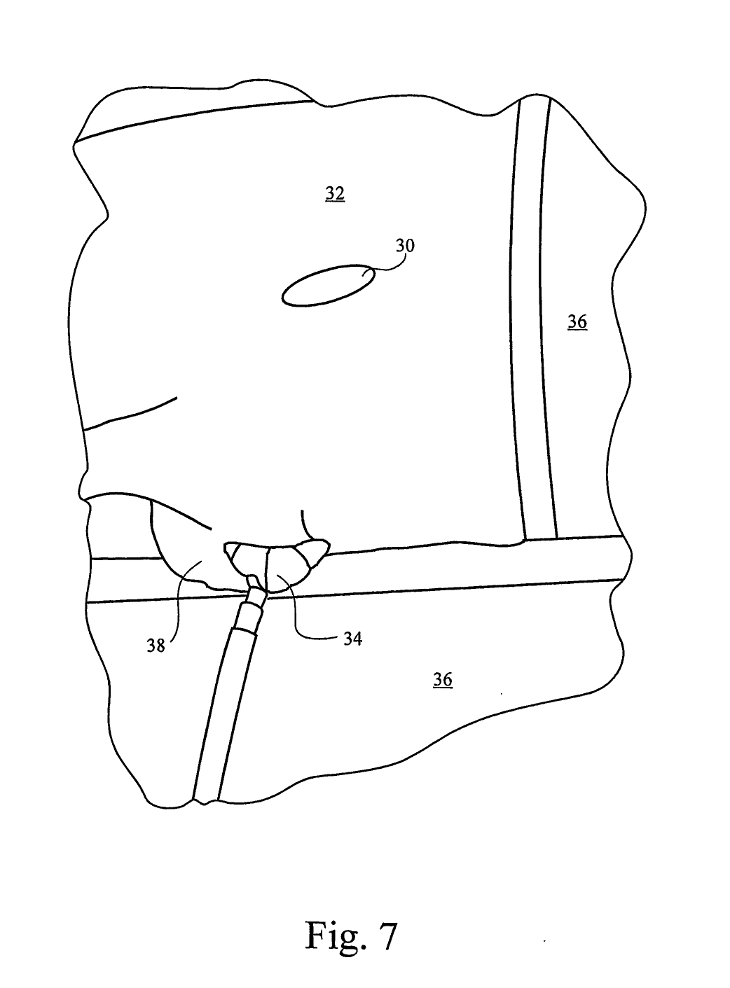

Referring now to FIGS. 3-7, in FIG. 3 the first step in the orchidopexy procedure is shown where an incision 30 is made in the abdominal wall 32 in the region of the undescended testis with the testis 34 located externally of the patient's body. In the drawing of FIG. 3 the tunneller body and tunneller head are shown on top of the patient's body lying in a position corresponding to the subcutaneous tunnel to be formed by the tunneller. The area 36 represents a surgical sheet covering the patient's body not involved in the surgical procedure. FIG. 4 shows the tunneller inserted in the incision 30 with the tunneller head moved to a position within the scrotum 38 to form a subcutaneous scrotal pouch (dartos pouch) 40. In FIG. 5 a second incision 42 is formed in the scrotum in the region of the tunneller head so that the tunneller may be pulled through the patient's body with the undescended testis 34 attached to the other end of the tunneller by means of a surgical stitch as shown in the drawings of FIGS. 6 and 7. The surgical procedure being completed by releasing the testis from the end of the tunneller and anchoring it to the scrotum prior to closing the incisions previously formed.

Although aspects of the invention have been described with reference to the embodiments shown in the accompanying drawings it is to be understood that the invention is not limited to these precise embodiments and that various changes and modifications may be effected without further inventive skill and effort.

1. An orchidopexy tunneller comprising an elongate body having a tunnelling head at one end thereof and a testicle attachment means at the opposite end thereof for attachment to a testicle to be repositioned in the human body by means of a surgical stitch during orchidopexy surgery.

2. A tunneller as claimed in claim 1 wherein the attachment means comprises an aperture provided in the end of the body opposite the tunneller head.

3. A tunneller as claimed in claim 1 wherein the elongate body is arcuate.

4. A tunneller as claimed in claim 1 wherein the tunneller head is of a shape and size representative of a human testis.

5. A tunneller as claimed in claim 1 wherein the tunneller is of a titanium or titanium alloy material.

6. A kit of orchidopexy tunnellers as claimed in claim 1 wherein the tunnelling head of each tunneller corresponds to a different size of testis.

7. A tunneller as claimed in claim 1 wherein the tunneller is a one-piece orchidopexy surgical tunneller.

8. A tunneller as claimed in claim 1 wherein the tunneller is a uni-directional orchidopexy tunneller.

9. A method of orchidopexy surgery on the male human or animal body, the said method comprising the steps of:
i) creating an incision in the abdominal wall of the body being treated in the region of the undescended testis;
ii) inserting a subcutaneous tunneller in the said incision and feeding the tunneller subcutaneously to the scrotum of the said body;
iii) forming a pouch in the scrotum to accommodate the said testis;
iv) creating an incision in the scrotum in the region of said pouch;
v) attaching the said testis to the tunneller at a position remote from the head of the tunneller;
vi) feeding the tunneller and the said attached testis through the subcutaneous tunnel previously formed until the said testis is positioned in the region of the said pouch;
vii) detaching the said testis from the tunneller;
viii) closing the said incisions with the testis positioned in the said pouch.

10. A method as claimed in claim 9 wherein the testis is attached to the said tunneller by means of a surgical stitch or mechanical fastening means.

11. A method as claimed in claim 9 wherein the testis is attached to the tunneller towards an end thereof remote from the said tunneller head.

12. A method as claimed in claim 9 further comprising the step of selecting a subcutaneous tunneller from a set of subcutaneous tunnellers having different size tunnelling heads, the selected tunneller having a tunnelling head which corresponds in size with the size of the said testis to be repositioned.

13. A method as claimed in claim 9 wherein the tunneller is fed through the said incision in the said scrotum to remove the tunneller from the body.
